(19) United States
(12) Patent Application Publication
(10) Pub. No.: US 2004/0260398 A1
(43) Pub. Date: Dec. 23, 2004

(54) Resorbable devices

(76) Inventor: David C. Kelman, Collierville, TN (US)

Correspondence Address: CHIEF PATENT COUNSEL, SMITH & NEPHEW, INC., 1450 BROOKS ROAD, MEMPHIS, TN, 38116, US

(21) Appl. No.: 10/776,409

(22) Filed: Feb. 10, 2004

Related U.S. Application Data
(60) Provisional application No. 60/446,210, filed on Feb. 10, 2003.
(60) Provisional application No. 60/446,241, filed on Feb. 10, 2003.

(51) Int. Cl. A61F 2/40

(52) U.S. Cl. 623/19.11; 623/20.11; 623/20.17; 623/23.23

(57) ABSTRACT

Resorbable devices and systems and methods utilizing the resorbable devices for adjunct stabilization of joint implants in the tubular bones of humans or animals during joint replacement procedures. The resorbable devices are at least partially resorbed by the body over a predetermined period of time, thereby allowing for bone ingrowth into a resulting void.

### CROSS-REFERENCE TO RELATED APPLICATIONS

The present application claims the benefit of U.S. Provisional Patent Application No. 60/446,210 filed Feb. 10, 2003, and U.S. Provisional Patent Application No. 60/446,241, filed Feb. 10, 2003.

### FIELD OF THE INVENTION

The present invention relates to systems, devices and methods for the stabilization of a joint implant during joint replacement surgery. More specifically, the present invention relates to systems for adjunct stabilization of prosthetic implants on bones of an animal or a human during joint replacement procedures. In one aspect, the present invention relates to system of stabilization of components of prosthetic joint implants on the bones of the animal or the human. More particularly, devices according to aspects and embodiments of the present invention provide temporary fixation of the prosthetic joint implants on the bones for a period of time necessary for a bone ingrowh on the surface of the implant to occur and provide permanent fixation. At the same time, devices according to aspects and embodiments of the present invention protect exposed bone tissue, particularly from undesirable contact with polymer particles, thus preventing osteolysis. In another aspect, the present invention relates to a resorbable device that is inserted in a cavity between a joint implant and a bone to prevent undesirable movement of the joint implant relative to the bone during healing and subsequent resorption of the device, thereby permitting bone ingrowth and joint stabilization. Devices according to aspects and embodiments of the present invention advantageously simplify attachment of prosthetic joint implants onto the bone, thus decreasing the risk of damage of the bone and soft tissues, reducing healing time, an decreasing the possibility of additional surgeries.

### BACKGROUND

Joint implants, also referred to as joint prostheses, joint prosthetic implants, joint replacements, or prosthetic joints, are long-term surgically implantable devices used to partially or totally substitute for diseased or damaged joints. Joints to be replaced include those such as hip, knee, shoulder, ankle, or elbow joints within the musculoskeletal system of a human or animal. Since their first introduction into clinical practice in the 1960s, joint implants have significantly improved the quality of life of numerous orthopedic patients. Both artificial hip joints and artificial shoulder joints are generally ball and socket joints, designed to match as closely as possible the function of the natural joint. Generally, the artificial socket is implanted in one bone, and the artificial ball articulates in the socket. A stem structure attached to the ball is implanted in another of the patient's bones, securing the ball in position.

The ball and socket joint of the human hip unites the femur to the pelvis, wherein the ball-shaped head of the femur is positioned within a socket-shaped acetabulum of the pelvis. The head of the femur or ball fits into the acetabulum, forming a joint which allows the leg to move forward, backward and sideways in a wide range. The acetabulum is lined with cartilage, which cushions the bones and allows the joint to rotate smoothly and with minimal friction. An envelope of tough ligaments connects the pelvis and femur, covering the joint and stabilizing it. Cartilage also makes the joint strong enough to support the weight of the upper body and resilient enough to absorb the impact of exercise and activity. A healthy hip allows the leg to move freely within its range of motion, while supporting the upper body and absorbing the impact that accompanies certain activities.

However, various degenerative diseases and injuries may require replacement of all or a portion of a hip using synthetic materials. Prosthetic components are generally made from metals, ceramics, or plastics or combinations of them.

Total hip arthroplasty and hemi-arthroplasty are two procedures well known within the medical industry for replacing all or part of a patient's hip and have enabled hundreds of thousands of people to live fuller, more active lives. A total hip arthroplasty replaces both the femoral component and the acetabular surface of the joint, so that both a femoral prosthesis and an acetabular prosthesis are required. A hemi-arthroplasty may replace either the femoral component or the acetabular surface of the joint. The purpose of hip replacement surgery is to remove the damaged and worn parts of the hip and replace them with artificial parts, called prostheses, which will help make the hip strong, stable and flexible again.

In hip replacement surgery, commonly referred to as total hip arthroplasty, a patient's natural hip is replaced by two main components, a stem member which takes the place of the femoral head, and an acetabular cup member which takes the place of the acetabular socket.

A conventional acetabular cup member may include a cup, a cup and a liner, or in some cases only a liner, all of which may be formed in various shapes and sizes. Generally, a metal cup and a polymeric liner are used. However, the liner may be made of a variety of materials, including polyethylene, ultra high molecular weight polyethylene and ceramic materials. The cup is usually of generally hemispherical shape and features an outer, convex surface and an inner, concave surface that is adapted to receive a cup liner. The liner fits inside the cup and has a convex and concave surface. The cup liner is the bearing element in the acetabular component assembly. The convex surface of the liner corresponds to the inner concave surface of the cup or acetabulum, and the liner concave surface receives the head of a femoral component. An acetabular cup may include a highly polished inner surface in order to decrease wear.

The stem and ball portion of the prosthesis may be a femoral prosthesis that generally includes a spherical or near-spherical head attached to an elongate stem with a neck connecting the head and stem. In use, the elongate stem is located in the intramedullary canal of the femur and the spherical or near-spherical head articulates relative to the acetabular component. Femoral prostheses used in total hip arthroplasty procedures may or may not differ from an endoprosthesis used in a hemi-arthroplasty. The femoral head of each type prosthesis is generally a standard size and shape. Various cups, liners, shells, stems and other components may be provided in each type arthroplasty to form modular prostheses to restore function of the hip joint.

During a total hip replacement, the surgeon will take a number of measurements to ensure proper prosthesis selection, limb length and hip rotation. After making the incision, the surgeon works between the large hip muscles to gain access to the joint. The femur is pushed out of the socket, exposing the joint cavity. The deteriorated femoral head is removed. In order to install the acetabular cup, the surgeon must prepare the bone by reaming the acetabular socket to create a surface suitable for accepting a cup, which may be held in place by bone cement, an interference or press fit, or it may have a porous outer surface suitable for bony ingrowth. The new acetabular shell is implanted securely within the prepared hemispherical socket. The plastic inner portion of the implant is placed within the metal shell and fixed into place.

Next, the femur is prepared to receive the stem. The hollow center portion of the bone is cleaned and enlarged, creating a cavity that matches the shape of the implant stem. The top end of the femur is planed and smoothed so the stem can be inserted flush with the bone surface. If the ball is a separate piece, the proper size is selected and attached. Finally, the ball is seated within the cup so the joint is properly aligned and the incision is closed.

The ball and socket joint of the human shoulder is prepared using a procedure similar to that described above. During a shoulder replacement operation, at least a portion of the proximal section of the humeral shaft is replaced by a metal prosthesis. This prosthesis generally consists of two parts: a stem that is mounted into the medullary canal of the humerus, and a head component connected in some manner to the stem. The head component replaces the bearing surface of the humerus and articulates within the glenoid cavity of the scapula to allow movement of the shoulder.

An arthritic humeral head (ball of the joint) may be removed and replaced with a humeral prosthesis. If the glenoid socket is unaffected, a hemiarthroplasty may be performed (which means that only the ball is replaced). The humeral component is made of metal and is usually press fit, but sometimes cemented, into the shaft of the bone of the humerus.

If the glenoid is affected, but conditions do not favor the insertion of a glenoid component, a non-prosthetic glenoid arthroplasty may be performed along with a humeral hemiarthroplasty. In this procedure, the glenoid shape and orientation are corrected, but a glenoid prosthesis is not inserted. The socket is reshaped by reaming and the prosthetic ball of the humeral component articulates with the reshaped bony socket of the glenoid.

In a total shoulder joint replacement, the glenoid bone is shaped by reaming and oriented, and then covered with a glenoid component. A small amount of bone cement is commonly used to hold the artificial glenoid socket in place.

During joint replacement surgery, also referred to as replacement arthroplasty, a joint implant is inserted into or otherwise attached to a bone that has been prepared to receive the implant, and the implant is secured. Reliable stabilization, or fixation, is essential for the success of joint replacement. Movement of the implant relative to the bone often results in formation of a fibrous interface between the implant and the bone. The fibrous interface may cause further loosening and, ultimately, destabilization of the implant, thereby necessitating additional surgery or surgeries, commonly referred to as corrective or revision surgeries.

Several methods of stabilizing joint implants, or components of joint implants, on or in a bone are known. One common method of stabilization is to permanently affix the joint implant to the bone using a bone cement. Stabilization with bone cement requires the drilling of oversized holes in the bone, which are filled with the cement prior to insertion of the joint implant. The implant is inserted into the cement-filled bone and allowed to harden. Unfortunately, implants that have been cemented are prone to loosening and are extremely difficult to remove when replacement is required. Furthermore, the cementing process requires difficult preparation of the bone surface. Moreover, cementing has been shown to be associated with increased risks of embolic events and intraoperative pulmonary impairment (Pitto, et al., (1999) J. Bone. Joint. Surg. Am. v. 81(6):831-43).

Other common methods of stabilizing a joint implant are cementless methods, which include stabilization by interference or press fit, stabilization by various structures, and other methods. For example, various stabilizing devices or structures, such as pegs, screws, or fins, protrude from the implant and are used to attach the prosthetic joint to the bone. The main disadvantage of this type of the cementless method is that the protruding structures frequently create stress patterns in the bone. These stress patterns produce undesirable bone remodeling that can lead to destabilization of the implant. In another type of cementless method, often referred to as biological fixation, the implants are covered, coated, or enveloped with a porous surface material, such as a polymer or a ceramic material, that allows bone growth into the surface of the implant. This growth, commonly referred to as “bone ingrowth”, stabilizes the implant. While less prone to the destabilization problems encountered when implants are stabilized by other methods, implants stabilized by biological fixation must remain completely secure for three to eight weeks after the implant surgery to allow sufficient bone ingrowth to occur. Any movement of the implant within the bone during these first few weeks after surgery results in the formation of a fibrous interface between the implant and the bone that prevents bone ingrowth and the desired stabilization effect.

When a prosthetic joint installed during primary joint replacement surgery deteriorates, loosens, destabilizes, or otherwise becomes problematic, the joint must be removed and replaced with a new joint during a subsequent (or revision) surgery. Stabilization of a new joint implant during a revision surgery is particularly challenging. As a result of the prior removal of large amounts of bone tissue during the first joint replacement surgery, cavities often exist between the new implant and the bone to which the new implant is being attached, thereby making it impossible to achieve a tight fit between the new implant and the bone. It is advantageous to fill these cavities and provide adjunct stabilization of the implant relative to the bone for several reasons. First, undesirable bone remodeling may occur if stress distribution after revision surgery changes. Thus, it is desirable to maintain the load transfer in a bone after revision surgery as similar as possible to that existing with the primary implant. Second, it is desirable to distribute stress patterns in the bone to help bone reconstitution and avoid risk of bone fracture. Third, the filling of cavities helps minimize stress levels in the implant itself, thereby reducing the risk of implant fracture. Additionally, when utilizing a biologically stabilized implant, the cavities should be filled to avoid undesirable movement of the implant relative to the bone, thereby preventing formation of a fibrous interface to allow proper bone ingrowth and permanent fixation of the implant. Moreover, cavities should be filled to prevent the exposure of unprotected bone tissue. Exposed bone tissue is susceptible to infection and is accessible to harmful polymer particles, which often form as a result of the “shedding” of the polymer-coated, articulating surfaces of joint implants. These polymer particles may cause localized osteolysis, a time-dependent process that arises from an inflammatory reaction caused by the particulate debris of polymer coatings composed of polymers such as polyethylene.

One particularly challenging joint implant stabilization scenario is found in hip replacement revision surgery. During this surgery, the proximal aspect of the femur resembles the shape of an ice cream cone as a result of previous surgeries. Therefore, during revision hip replacement surgery, it is impossible to adjust both the distal and proximal aspects of the femur to fit the implant. When a distally fixed hip implant is installed into a femoral canal, the implant passes through the ice cream cone-shaped proximal aspect of the femur and is secured to a distal aspect of the femur. Due to the shape, a cavity often remains between the implant and the cortical bone tissue in the proximal aspect of the femur. It is desirable to fill the cavity to distribute some of the stress between the distal and proximal femur in order to promote bone reconstitution in the proximal femur. In addition, the filling of the cavity provides additional stabilization of the implant, thereby decreasing the risk of it becoming loose.

Another challenging scenario is, an example of hip revision surgery when an acetabulum needs reamed excessively until a substantial portion of it is hemispherical in order to install an acetabular component of a hip prosthesis. Often, achieving a desired shape is still impossible, and a non-optimal bi-lobed configuration of an acetabular component is utilized, such as that in the acetabular components offered by DePuy Orthopaedics (Warsaw, Ind.), or Johnson & Johnson, (New Brunswick, N.J.), or a revision acetabular cage is used. During revision knee replacement arthroplasty, femoral or tibial components are often combined with metal augments, which are wedges or blocks of metal to make up for the lost bone and fill the gaps.

However, the shape of the cavities between a joint implant and a bone is often irregular and cannot be filled by standardized metal implants. To circumvent this problem, the technique of allografting, or packing allograft bone into bone cavities, followed by the introduction of bone cement, is often utilized. The allograft bone is crushed, morselized, or fashioned into shapes suitable for packing the cavity, the shapes are packed into the cavity, and bone cement is added. This allografting technique is prone to all of the problems described above that are associated with cementing methods. Specifically, during any subsequent revision surgeries, removal of the allograft and additional bone tissue is required. This cumulative bone loss creates a natural limit to a number of revisions that can be performed. Restoration of bone tissue, or stock, is limited or does not occur when allografts are used. Moreover, bone necrotization may occur and persist in the allografts. In addition, bone remodeling and revascularization are very slow (Tagil (2000) Acta Orthop. Scand. Suppl., v. 290:1-40).

Morselization of the allograft material may promote bone remodeling and restoration by causing the release of growth factors present in the graft, and the morselized material may be impacted to make it easier for the ingrowing bone to climb up into the graft (Tagil (2000) Acta Orthop. Scand. Suppl., v. 290:1-40). However, when allograft bone is being packed into the cavity, significant force is utilized. The use of such force increases risk of bone fracture and trauma. If the allograft material is not morselized, but is fashioned into preformed shapes, the shapes available often do not fit properly into the cavity to be filled. Finally, the use of allografts, in general, carries increased risks of disease transmission and graft rejection. One alternative to allografting is to collapse the adjacent bone around the implant and cable the bone onto the implant. However, this procedure has been associated with bone degradation.

Permanent, such as, metallic, fixation devices, must be removed at time of revision surgery. In the case of screws, there are times when they are difficult to remove, which is time consuming to the surgeon. An added disadvantage is that such devices, particularly screws, may fracture, with the resulting remnants causing tissue damage. Also available are spikes, pegs, or fins, or any combination thereof, that are driven into the bone, for example, for fixation around the periphery or the dome of acetabular cups, however, upon their removal, a hole or cavity is formed that still must be filled with bone graft during revision surgery.

A modular peg is currently available for fixation of an acetabular component of a hip implant. The modular peg can be inserted for fixation after the acetabular cup was implanted. Due to it being broad compared to the root diameter of a screw, the peg provides better rotational stability than the screw. Moreover, the peg seals a connection between the acetabular cup and the peg, thereby preventing exit of debris through this connection. During revision surgery, the peg can be removed prior to removal of the cup, which allows curved osteotomies, or gouges, to be passed around the outside of the acetabular shell during surgery, thus simplifying the procedure. Still, the modular peg results in a cavity in an acetabular bed that requires packing with bone graft or other materials.

Temporary fixation structures manufactured of resorbable, degradable, or temporary, materials are gradually resorbed by the tissue after the installation. The resulting bone cavity is gradually replaced by re-growing bone. A variety of biodegradable devices for temporary fixation of joint implants, all of which, however, suffer from a variety of shortcomings. Bone screws manufactured of materials that are resorbed by the body are available. While they do not require removal after the need for fixation passes, they are of limited mechanical strength. When bone screws are used for fixation of an acetabular component of a hip implant, fixation is often lost prematurely, thus not allowing adequate time for the bone ingrowth to occur, and resulting in destabilization of the implant on the bone. In general, bone screws, including those made of resorbable materials, are known to back out of the bone, press against polymer components of a joint implant, create dents, and generate polymer particles. Moreover, degradation, or resorption of the screw material occurs faster than bone re-growth, thus leaving exposed cavity, which is also prone to osteolysis, particularly in the cases where polymeric prosthetic surfaces are employed that may generate particles during operation of the prosthesis. Generally, a challenge is to adjust the rate of degradation of such a temporary fixation device to correspond to the rate of bone tissue regrowth, thereby avoiding presence of a bone cavity, or exposed bone tissue, prone to infection or osteolysis. Covers, or seals, to cover screw holes in the bone, are available, however, there use requires additional fitting and installation steps during surgery.

In general, many of the currently available methods and devices for adjunct stabilization of implants, such as impaction allografting or the collapsing and cabling of bone, require the presence of sufficient amounts of high quality bone tissue in the bone to which the implant is being attached. When bone tissue is lost, due to disease or a pathological condition or for other reasons, the constructs become unstable. Persons with thin or fragile bones, such as osteoporosis patients, avascular necrosis patients and patients with metastatic bones, are especially in need of joint implants. However, their bone tissue is often not sufficiently strong for the stabilization of joint implants without adjunct fixation. Therefore, currently available adjunct stabilization devices and methods fail to satisfy the requirements of patients who are most in need of joint implants.

Thus, there is a need for systems and devices that provide reliable adjunct stabilization of joint implants yet allow for reestablishment of bone, bone ingrowth or restoration of bone stock. Particularly, there is a need for methods that provide additional stabilization of implants in tubular bones. Specifically, implants are needed that permit and promote bone regrowth in the cavities between the implant and the bone cortex. This need is particularly urgent during revision arthroplasties, such as revision hip replacement arthroplasty, when a large amount of bone tissue has been removed during prior surgeries and it is impossible to tightly fit an implant into a bone without leaving a bone cavity, or in situations when disease and pathological conditions reduce the amount and quality of bone tissue available for fitting.

Devices and systems for adjunct stabilization are needed that allow for bone restoration, are easily adaptable to a variety of local conditions, reliably stabilize the implant, maintain the load transfer, and distribute stress patterns in the bone in a manner that promotes bone reconstitution, reduces the risk of bone fracture, reduces stress levels in the implant itself and reduces the risk of implant fracture. Suitable devices and systems are needed that generally reduce undesirable movement of the implant relative to the bone for a required period of time, thereby preventing formation of a fibrous tissue at the bone-implant interface. There is a particular need to temporarily and reliably stabilize uncemented joint implants to allow for bone ingrowth to occur on the surface of the implant and permanently stabilize the implant on the bone. At the same time, the devices and structures for adjunct stabilization are needed that would allow foregoing additional surgical procedures necessary to remove the devices from the body once the need in the stabilization, or fixation, passes, and, preferably, would allow avoiding the removal of the temporary fixation devices and structures during any required revision surgeries altogether, thus avoiding the risks of tissue damage associated with such removal and other surgical complications.

Moreover, systems and devices are needed that protect exposed bone tissue from undesired contact with particles and infectious agents after installation of the implant, thereby reducing the risks of infection and osteolysis. Additionally, devices and systems are needed that are sufficiently inert and stable in the human body, possess adequate mechanical and chemical properties to stabilize a joint implant in tissues and reliably remain in the tissues without causing undesirable side effects, such as degradation, undesired bone or soft tissue redistribution, or mechanical damage. The needed devices and systems should also provide adequate stabilization for a sufficient period of time to allow bone ingrowth to occur. Also desirable are systems that can serve as carriers for advantageous biologically active molecules, such as growth factors or antibiotics.

Also, stabilization devices and systems are desired that simplify removal of the implant during any required subsequent revision surgeries, thereby decreasing bone degradation and the risks of tissue damage associated with such removal, as well as other surgical complications. In general, temporary fixation devices and systems are needed that are readily available to a surgeon, easy to use, minimize tissue damage, and simplify any subsequently required surgical procedures. Temporary fixation devices and systems are needed that are versatile, allow for faster healing with fewer complications, require less immobilization, are easy to use and manufacture, and are inexpensive to produce and operate.

### SUMMARY

Resorbable devices and systems and methods of use thereof for adjunct stabilization, or fixation, of orthopedic implants are described herein. In a preferred embodiment, systems, devices, and methods for adjunct stabilization of joint implants, such as hip, knee, or shoulder implants, or various components thereof are provided.

In one aspect, the resorbable stabilization systems, devices and methods are advantageously used to ensure temporary stability of an implant or components thereof after being implanted, secured, embedded or surgically inserted within a body of a human or animal. In another aspect, the adjunct stabilization devices and systems described herein allow for restoration of bone tissue, reestablishment or regrowth of bone tissue or bone; also referred to herein as bone ingrowth, bone restoration, or restoration of bone stock. The present invention combines the foregoing aspects in devices that are installed during joint replacement surgeries in spaces, or cavities, between a joint implant and bone. After installation, the devices are gradually resorbed by the body of the human or animal in which the devices are installed, thereby allowing bone tissue to regrow in the space or spaces previously occupied by the resorbed devices.

In one more aspect, during a predetermined period of time required for restoration of bone, the resorbable devices and systems provide adjunct stabilization to the implant, beneficially maintain the load transfer and distribute stress patterns in a bone (thereby aiding in bone reconstitution and reducing a risk of bone fractures), reduce stress levels in the implant itself (thereby reducing the risk of implant fracture), and generally reduce undesirable movement of the implant relative to the bone, thereby preventing undesirable side effects, such as formation of a fibrous tissue at the interface between the implant and the bone. In preferred embodiments, the resorbable adjunct fixation systems and devices also protect exposed bone tissue from undesired contact with particles and infectious agents after installation of the implant, thereby reducing the risks of infection and osteolysis. Additionally, due to their relatively low rigidity when compared to conventional stabilization structures such as metal spacers, the resorbable devices described herein are capable of being installed and used in cavities exhibiting a shape that conventional devices are unable to fill.

In another aspect, the resorbable adjunct fixation systems and devices described herein are adaptable for installation into existing spaces in the bone and permit bone regrowth into those spaces, thereby increasing the amount of healthy bone tissue. In contrast to conventional stabilization systems, which often require removal of bone tissue during implant installation and additional bone tissue removal during revision surgery, the resorbable adjunct fixation devices described herein reduce bone degradation and the risks of tissue damage associated with implant removal, as well as reducing other surgical complications.

In one more aspect of the present invention, the resorbable adjunct fixation systems and devices described herein are especially suitable for stabilization of cementless joint implants, wherein temporary stabilization is desirable while bone ingrowth occurs on the surface of the implant and provides permanent stabilization. In one variation on the preferred embodiments of the current invention, a resorbable device is provided, which comprises a resorbable, or degradable, component, and an integral permanent component, which seals the exposed edge of the temporary fixation device after it is inserted into the bone and prevents undesired access of particles, compounds, and infectious agents to exposed bone tissue, thereby reducing a possibility of infection, osteolysis, or other damage to the bone tissue, and creating favorable conditions for bone and soft tissue growth and healing. A temporary and a permanent component of the device according to this preferred embodiment are integrally unified in a hybrid resorbable device, or structure, which simultaneously and advantageously provides temporary fixation of an implant and permanent protection of exposed bone tissue.

In general, the resorbable devices according to aspects and embodiments of the present invention allow for simplified fitting and installation as compared to conventional systems. Also, when compared with conventional systems and devices, the temporary fixation devices and systems described herein are more versatile, allow for faster healing with fewer complications, require less immobilization, and are less costly to produce and operate.

Additional features, objects, and advantages of the invention will become apparent from the detailed description of the preferred embodiments.

### BRIEF DESCRIPTION OF THE DRAWINGS

FIG. 1 is a top view of a proximal femur during revision hip replacement surgery showing an example of a distally secured hip implant in which the implant is secured distally with no proximal support.

FIG. 2 is a top view of a proximal femur during revision hip replacement surgery in which the implant is secured distally and resorbable spacers are inserted into cavities between the implant and bone to wedge the implant to the proximal femur.

FIG. 3 is a schematic representation of a hybrid resorbable peg.

FIG. 4 is a schematic representation of a hybrid acetabular cup.

### DETAILED DESCRIPTION OF PREFERRED EMBODIMENTS

Resorbable devices for the adjunct stabilization of orthopedic implants in a human or animal are provided. Systems and methods employing the resorbable devices are also described herein. The systems, devices and methods provided herein are preferably useful during the insertion of hip, knee, shoulder or elbow implants in joint replacement surgery.

### Resorbable Devices

The resorbable adjunct stabilization devices, or spacers, according to certain aspects of the present invention comprise one or more resorbable, bioerodible, or degradable material. Upon installation of the implant, gradual resorption of the spacers takes place, thereby making space available for bone ingrowth. Moreover, by providing mechanical tension, or loading, of bone tissue, the spacer promotes bone ingrowth. The biodegradable spacers are at least partially manufactured of biodegradable materials, including, but not limited to, materials of a polymeric nature, or polymers. The term “biodegradable” is used interchangeably with the terms “bioabsorbable”, “bioresorbable”, “resorbable”, “degradable”, “erodible”, or “bioerodible”, and these terms are used to characterize materials that gradually disintegrate after implantation into a human or an animal.

Biodegradable materials used in the certain aspects and embodiments of the present invention are preferably biocompatible, meaning not eliciting unresolved inflammatory response or demonstrating extreme immunogenicity or cytotoxicity, by an intact material, any of its unreacted components, or degradation products. Biodegradable materials used in the certain aspects and embodiments of the present invention preferably possess desirable combination of mechanical properties, such as those beneficial for providing proper support in the early stages of healing, and including, but not limited to, desirable compression, tension and torsion, characteristics. In general, the resorbable material is chosen such that it possesses a combination of desirable mechanical properties that are simultaneously advantageous for easy installation and for adequate fixation of the implant for a sufficient period of time necessary for healing and bone regrowth to occur. Advantageous mechanical properties include a sufficient amount of rigidity to prevent movement of the implant in the bone, and pliability to permit conformation of the implant to all or a portion of the unique shape of the cavity, thereby maximizing contact between the resorbable spacer, the implant and the bone.

Biodegradable materials used in the certain aspects and embodiments of the present invention are preferably beneficial for promotion of tissue formation, with properties such as an amount of void space and degradation chosen to encourage tissue growth and vascularization, if appropriate, within the material. Degradation rate may be coupled to the rate of bone tissue formation so that neither the load-bearing capabilities of the tissue, nor tissue regeneration are compromised. Accordingly, degradation rate of biodegradable materials according to a preferred embodiment of the present invention and a devices manufactured thereof are such so that to ensure adequate time for growth of bone tissue into a void, space, or cavity between a bone and a joint implant. In a preferred embodiment of the present invention, a device is at least partially resorbed over a predetermined period of time, less than approximately several years, preferably less than approximately two years. The predetermined period of time is chosen depending on a particular application and can be, for example, less than approximately one year, or less than approximately three months, or less than approximately twelve weeks, or from approximately three to approximately eight weeks. As with all implanted materials, biodegradable materials according to certain aspects and embodiments of the present invention are sterilizable to prevent infection. Sterilization, however, does not substantially interfere with the bioactivity of the material or alter its chemical composition, and does not substantially affect its biocompatibility or degradation properties.

The resorbable materials according to certain aspects and embodiments of the present invention include, but are not limited to, polymeric materials, such as poly-alpha-hydroxy acids, polylactide and polyglycolide, including their copolymers, poly-(D,L-lactide-co-glycolide) and polyglycolide-co-trimethylencarbonate; stereopolymers, such as poly-(L-lactide) or poly-L-lactic acid (PLA), poly-(L-CO-D,L-lactide) and poly-(D,L-lactide), polyglactin acid (PGA), a combination thereof (PLA/PGA) or any derivative, combination, composite, or variation thereof, poly-(D,L-lactide-co-glycolide) (PDLLA-co-PGA), poly-(L-lactide) (PLLA), poly-(L-lactide) (PLLA), polyglycolide-co-trimethylencarbonate, (PGA-co-TMC), poly-(L-CO-D,L-lactide), poly-(D,L-lactide), (PDLLA). The use of slow degrading and highly crystalline polymers, such as poly-(L-lactide) and poly(L-CO-D,L-lactide) stereocopolymers with a low D,L amount, amorphous polymers, such as poly-(L-CO-D,L-lactide) stereocopolymers with a high D,L amount and the purous poly-(D,L-lactide), or fast-degrading copolymers, such as poly-(D,L-lactide-co-glycolide) or polyglycolide-co-trimethylencarbonate, is envisioned and falls within the scope of the present invention. The use of injectable or crosslinkable polymers, including, but not limited to, photopolymerizable and chemically polymerizable polymers and polymers that harden in situ, is also encompassed by the present invention, including but not limited to the use of polymers of sebacic acid (SA), alone, or copolymers of SA and 1,3-bis (p-carboxyphenoxy) propane (CPP), or 1,6-bis (p-carboxyphenoxy) hexane (CPH), or poly(propylene fumarate) (PPF). Resorbable materials for use in the devices according to the embodiments of the present invention are not limited to the foregoing and includes any fully or partially degradable or erodible in a body chemical composition or material suitable for use in the devices according to the embodiments of the present invention, including but not limited to carbohydrates and derivatives thereof, such as such as cellulose or hyaluronic acid, in resorbable devices of the embodiments of the present invention. Modifications of polymeric materials to adjust their structural, mechanical or chemical properties, or facilitate biological responses in tissues is envisioned and falls within the scope of the present invention.

In a preferred embodiment of the system provided herein, the resorbable adjunct stabilization devices incorporate bioactive molecules that promote beneficial processes, such as healing, regeneration, bone regrowth and mineralization, and discourage undesirable processes, such as infection or inflammation. The bioactive molecules for incorporation into the adjunct stabilization devices include, but are not limited to, antibiotics; growth factors, including, but not limited to, insulin-like growth factors (IGF-I & II), transforming growth factors (TGFbs 1-3), fibroblast growth factors acidic and basic (aFGF & bFGF), platelet derived growth factor (PDGF), and bone morphogenetic proteins (BMPs); interleukins (IL), such as IL-1 b, IL-3 (Multi CSF), IL-4, IL-6, and IL-8; tumor necrosis factors TNF alpha and TNF beta; interferons (IFNs); colony stimulating factors; hormones, including but not limited to steroids, such as estrogen, and peptide hormones; anti-inflammatory molecules, including non-steroidal anti-inflammatory molecules; or any combination or variation thereof. The bioactive molecules are incorporated into resorbable adjunct stabilization devices according to any suitable method, including but not limited to, caging, impregnation, complexing, or chemical bonding, including the use of covalent and non-covalent bonds. Examples of beneficial modification with biologically active molecules include modification GRGD (Gly-Arg-Gly-Asp) peptide sequence to encourage host cell attachment and migration, and encapsulation of growth factors, such as TGF-1, which acts to direct cell migration and differentiation.

Devices and systems incorporating both resorbable and non-resorbable materials are also envisioned and fall within the scope of the present invention. Such composite devices and systems allow for control over the rate and extent of resorption during healing. Also envisioned and falling within the scope of the present invention is a composite containing a plurality of resorbable materials. The composite possesses a desirable and advantageous combination of mechanical and resorption properties appropriate for a particular device or method according to embodiments described herein.

### Resorbable Spacers

In one preferred embodiment, the systems, methods and devices are advantageously used during joint replacement surgery when a joint implant cannot be connected to a bone without leaving a cavity, space or void, between the implant and the bone. In one example, use of the devices is advantageous when a large amount of bone tissue has been removed during prior surgery or a bone defect is present. The resorbable adjunct fixation devices, or resorbable spacers as they are commonly referred to herein, are inserted into the cavity, thereby filling or at least partially filling the void. The resorbable spacers provide adjunct stabilization of the implant by occupying all or a portion of the open space between the cavity and the bone, preventing movements of the implant relative to the bone, and maintaining an even load of stress, force or pressure on the bone.

The resorbable spacers maintain adequate fixation of the implant to the bone for a sufficient amount of time to allow bone ingrowth into the cavity. Meanwhile, the presence of the spacers permits beneficial loading of the bone tissue and distributes mechanical tension therein. After the prosthetic implant has been stabilized with the resorbable spacers for a predetermined period of time, the degradable component of the spacers is gradually resorbed by the bone tissue. The regrowing bone gradually fills or at least partially fills the voids created by the erosion, or biodegradation, of the resorbing material, thereby eliminating or reducing the voids and providing a tighter, permanent fit of the implant in the bone.

In a particularly preferred embodiment, the resorbable spacers are used during revision hip replacement surgery to at least partially fill a cavity in a proximal femur during installation of a distally loaded hip implant in the femur, thereby acting as resorbable spacers between the implant and the proximal cortex of the femur.

The resorbable adjunct stabilization devices are preferably manufactured in a variety of shapes, including but not limited to, spheres, ovals, rectangles, trapezoids, triangles, conical shapes, tubes, rods, horse shoe or U-shapes, rings, toroids, wedges, spikes, shapes having elongated components or members, shapes having tapered or non-tapered edges, and the like. The resorbable devices are adapted for insertion into cavities of bones or other tissues. It is to be understood that, at the time of surgery, a surgeon selects a shape best fit for a task, or situation, at hand. The resorbable devices optionally contain a resilient material that is moderately soft or pliable. Such a device will not require an exact fit prior to insertion into the cavity as it may be slightly compressed or impacted during insertion into the cavity to ensure a tight fit. Alternatively, the device optionally contains a material having increased pliability at temperatures in excess of normal body temperature. Such a device may be heated to a predetermined temperature prior to insertion to achieve maximum flexibility and conformity to the cavity, and the flexibility dissipates upon cooling down to body temperature. Moreover, the resorbable devices can be shaped or trimmed by the surgeon, for example with a burr, before or during surgery as desired. These features advantageously distinguish the resorbable devices provided herein from conventional adjunct stabilization devices, such as metallic spacers.

### Methods of Use of Resorbable Spacers

Also included within the scope of the present invention are methods of installing joint implants using the resorbable adjunct stabilization devices described herein. In accordance with the methods, a tubular bone is prepared according to conventional methods, a joint implant installed into the bone canal, and one or more resorbable adjunct stabilization devices inserted into a cavity between an implant in a bone. In one embodiment, the adjunct stabilization devices are wedged, forced, pushed, or impacted into the cavity during installation, thereby ensuring a tight fit of the device in the cavity. In another embodiment, one or more resorbable devices are inserted into the cavity between the implant and bone, the outer surface of the bone is surrounded with an orthopedic cable, and the cable is tightened, thereby tightening the bone against the resorbable material, which, in turn, is tightened against the implant. A combination of the above embodiments is also envisioned and falls within the scope of the present invention.

The use of the resorbable adjunct stabilization devices in conjunction with any other stabilization techniques, including but not limited to, allografting, cementing, or packing a bone cavity with calcium sulphate or phosphate materials is envisioned and falls within the scope of the present invention. The use of the resorbable devices in combination with other stabilization devices and materials is, in one aspect, advantageous in that it reduces reliance on each device and material and amount thereof, thereby allowing the surgeon to vary and adjust the amounts in accordance of requirements of a particular situation. In another aspect, the use of the resorbable adjunct stabilization devices in conjunction with any other stabilization devices, such as allografting, allows for beneficial loading of the stabilization devices, thereby promoting bone regrowth, or remodeling. For example, the resorbable device optionally contains or is inserted into the cavity in combination with allograft bone or resorbable granules. The resorbable granules are composed of the materials described above with regard to the resorbable device.

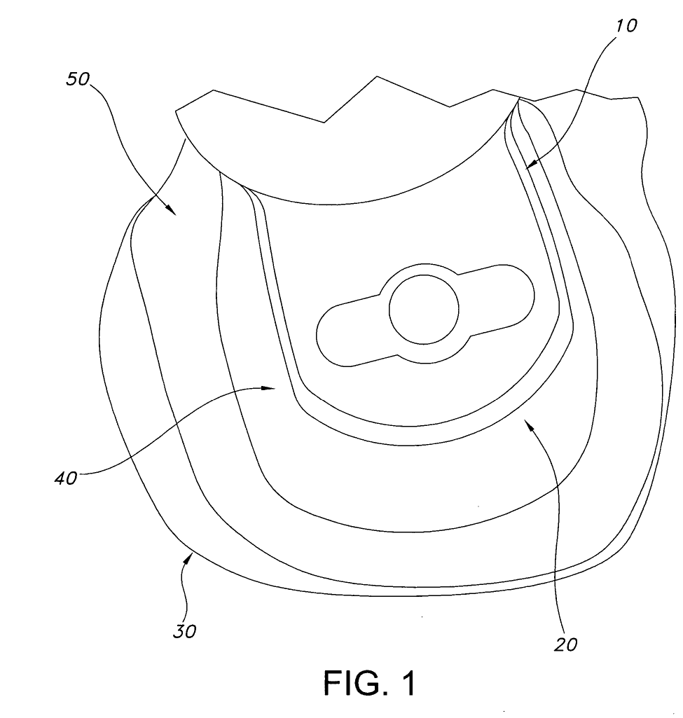

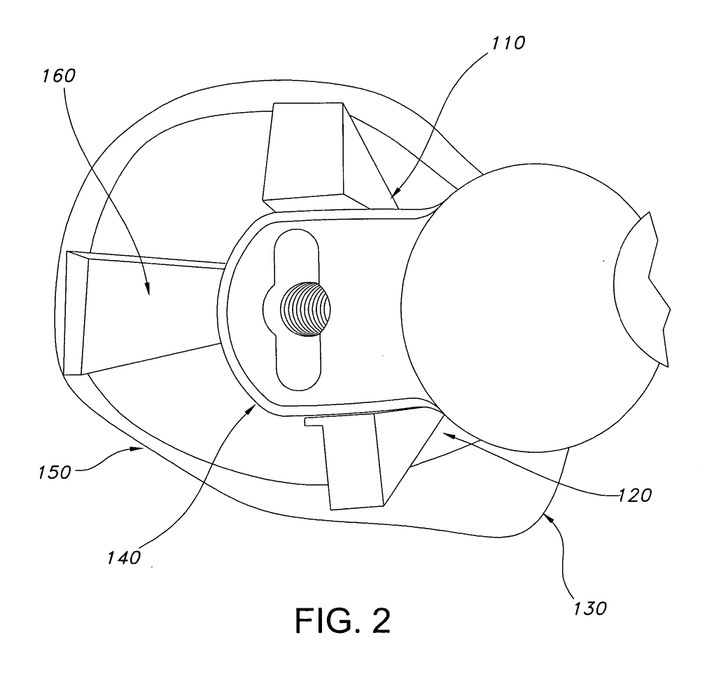

In a preferred embodiment, the resorbable adjunct stabilization devices are used in installation of a distally fixed hip joint implant during revision surgery. For example, during initial hip replacement surgery, a proximal femur is shaped to accept a joint implant in which a portion of the femoral bone tissue had been previously removed. During revision surgery, the proximal femur is often conically shaped (such as in the shape of an ice cream cone), which makes fitting of an implant difficult. FIGS. 1 demonstrates the cavity created during conventional hip replacement surgery as follows. A distally fixed implant (10) is utilized during revision surgery. The implant is inserted through the intramedullary canal (20) of the femur (30), passed through a proximal aspect of the femur, and fitted and secured into a distal aspect of the femur. In the proximal aspect of the femur, a large cavity (40) exists between the femoral stem of the implant (10) and the proximal cortical femoral bone (50). FIG. 2 shows the use of the resorbable spacers described herein to stabilize an implant during hip replacement surgery. A distally fixed implant (110) is inserted through the intramedullary canal (120) of the femur (130), passed through a proximal aspect of the femur (130), and fitted and secured into a distal aspect of the femur (130). In the proximal aspect of the femur (130), a large cavity (140) exists between the femoral stem of the implant (110) and the proximal cortical femoral bone (150). A degradable spacer (160) is placed within the cavity (140), or space, between the implant (110) and the proximal cortical bone (150). The spacer, is simple, easy to use, readily available “off the shelf”, and manufactured of sufficiently stable, or long-term, but, ultimately, resorbable, or degradable, materials. Additional spacers are placed within the space remaining between the implant and the proximal femur.

### Hybrid Resorbable Devices

In accordance with another preferred embodiment of the present invention, a hybrid resorbable device is provided, which comprises a resorbable, polymeric component, and a permanent component, also referred to as non-resorbable or non-degradable. The resorbable component comprises one or more resorbable material. The non-resorbable component is manufactured of one or more permanent material. Devices and structures according this preferred embodiment of the present invention simultaneously and advantageously utilize properties of resorbable and non-resorbable materials and overcome the disadvantage of the fixation devices manufactured of entirely permanent or entirely resorbable materials.

In one preferred embodiment, the resorbable component of the hybrid resorbable device has a configuration adapted for convenient insertion into bone tissue, including but not limited to screw, peg, pin, needle, or fin configuration, or any combination or variation thereof.

According to certain aspects and embodiment of the present invention, during joint replacement surgery, a joint implant is surgically inserted on a bone according to conventional procedures, and temporary fixation devices according to embodiments of the present invention are used to stabilize the implant on the bone, After the joint implant is installed, the resorbable component is gradually resorbed into the bone after attachment of an implant, wherein the non-resorbable component forms a protective covering, or seal, of the resorbable component, thereby protecting the any exposed bone tissue or cavities in the bone from exposure to undesirable particles or infectious agents.

Systems comprising hybrid resorbable devices according to certain aspects and embodiments of the present invention, and methods of use of the hybrid resorbable devices of the present invention and systems comprising thereof also fall within the scope of the present invention.

The use of devices and structures according to certain embodiments of the present invention is particularly advantageous during stabilization of implants with polymer components, such as polymer articulating surfaces, that shed polymer particles during operation. The devices and structures according to preferred embodiments of the present invention protect exposed bone tissue from polymer particles, thereby preventing osteolysis, or focal osteolysis, induced by the particles. According to certain aspects and embodiment of the present invention, during joint replacement surgery, a joint implant is surgically inserted on a bone according to conventional procedures, and temporary fixation devices according to embodiments of the present invention are used to stabilize the implant on the bone, wherein the resorbable component of the fixation device is fully inserted into the bone, and the non-resorbable component is fully or partially the outside of the bone. After the joint implant is installed, the resorbable component is gradually resorbed into the bone after attachment of an implant, wherein the non-resorbable component forms a protective covering, or seal, of the resorbable component, thereby protecting the any exposed bone tissue or cavities in the bone from exposure to undesirable particles or infectious agents.

A material of the nonresorbable component of the hybrid resorbable devices according to certain embodiments of the present invention is sufficiently inert in a body of a human or an animal and possesses mechanical properties necessary to provide a permanent protective cover of bone. In a preferred embodiment, a material for the non resorbable component is at least one of nonresorbable materials. In a preferred embodiment, a nonresorbable material is a metal, including but not limited to cobalt, chrome, or titanium, or any combination, composite, or alloy thereof. Resorbable and non-resorbable components and materials of the present invention are joined or united by any suitable method. In one embodiment of the present invention, the devices comprising a resorbable and a nonresorbable component are referred as composite.

The hybrid resorbable devices according to one variation on a preferred embodiment of the present invention are elongated members, such as pegs or fins, comprising at least one resorbable component and at least one permanent component. During implantation of a prosthetic implant, the resorbable component is inserted into a bone to fixate the prosthetic implant to the bone. The resorbable component is gradually resorbed into the bone after surgical insertion of the implant. The resorption period is such that adequate fixation is provided for a period of time necessary for bone ingrowth to occur and to fixate the prosthetic implant onto the bone, after which period the fixation provided by the resorbable device is no longer needed.

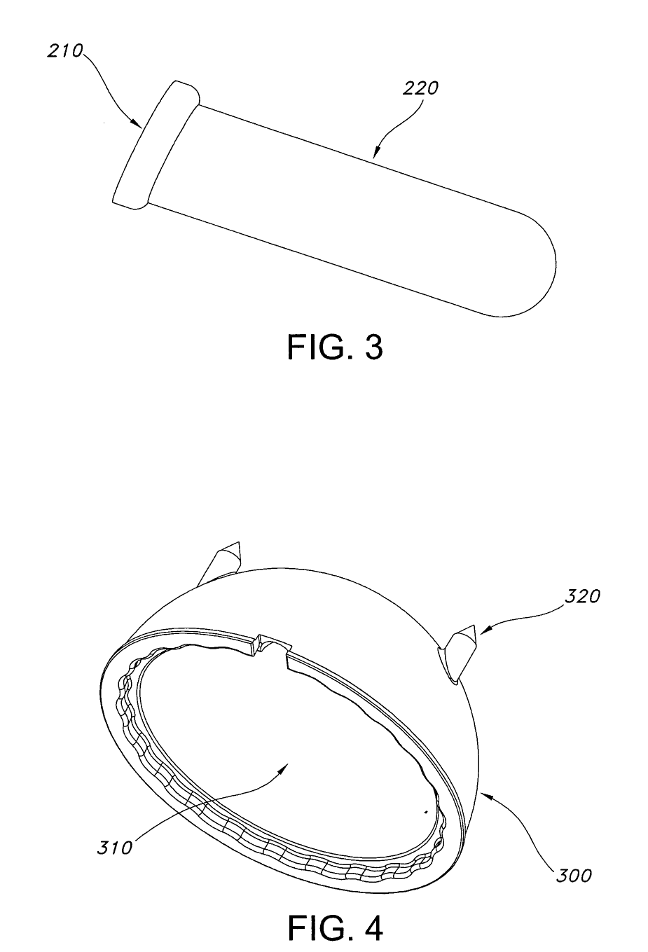

An example of device according to a variation on the preferred embodiment is a hybrid peg, or a reflection peg, shown in FIG. 3. The hybrid peg includes a shoulder portion comprising a metal component (210), which is instrumental in maintaining a water-tight seal once a peg is inserted into the bone, and at least partially resorbable peg portion (220), for maintaining for a required period of time, preferably for approximately at least twelve weeks, after insertion into the bone.

In certain embodiments, the hybrid resorbable device is ultrasonically welded, molded, heat pressed, by heating a resorbable component, a permanent component, or both, or otherwise attached, for example, by mechanical means, to a surface of a prosthetic implant, including but not limited to a tibial or a femoral component of a knee prosthetic implant, a glenoid component of a shoulder implant, or an acetabular cup component of a hip implant. The prosthetic implant stabilized with the hybrid resorbable devices comprises openings through which the fixation devices are inserted during surgery. According to a preferred embodiment of the present invention, the permanent component of the fixation device is a metallic piece, which, upon insertion of the device through the opening into the prosthetic implant into the bone, is flush with the surface of the prosthetic implant, securely anchored to the surface of the covers the opening and securely anchored to the surface of the prosthetic implant.

In one embodiment, a polymeric peg or fin or adjunct fixation devices is ultrasonically welded to the surface of a n acetabular cup, glenoid, tibial or femoral component. In FIG. 4, an acetabular cup (300) with a metal shell (310) and resorbable pegs, or spikes, (320) is schematically represented. The spikes are secured to the conventional metal shell. The hybrid acetabular cup is implanted by a method of implantation similar to conventional current surgical techniques. A resorbable material of the resorbable component begins to loose its mechanical properties several months after implantation, or wherever the need for fixation from the spikes no longer is necessary. Should revision surgery ever be necessary, the resorbable spikes would not adversely affect the ability of a surgeon, during revision surgery, to remove the hybrid device, as, by this time, bone, at least partially, fills the spaces left by the resorbable material, unlike when conventional metal spikes are used.

In another variation, a conventional implant in which holes, or openings, are present, such as those for metallic anchoring devices, is modified so that the permanent component of the hybrid device, such as a metallic piece component is designed and manufactures to be inserted into the opening, or function as the opening's cover, and is securely anchored to the shell, tibial plateau or glenoid component of the implant, thereby integrating the resorbable component into the cover.

In method of use of a hybrid resorbable device according to this variation on a preferred embodiment of the present invention, an acetabular component of a hip implant, comprising a screw opening, is inserted according to a standard method; comprising creating an initial cavity, installing the acetabular component, and inserting a hybrid resorbable device through the screw opening in the acetabular component until the component is fully seated in a bone. A permanent, such as metal, portion of the hybrid resorbable device is securely attached to the acetabular shell. This method is not limited to installation of acetabular components of hip implants, but is also used for tibial components, acetabular components, or any implants, where there is a hole, or an opening, or need for adjunct stabilization, or fixation.

Other variation on hybrid resorbable devices according to preferred embodiments of the present invention include, but are not limited to, various fixation, or stabilization, devices, such as, but not limited to, those similar to a “pop rivet”, drywall mounting screw, a Richards Flex Lock Peg, etc. In situations of revision surgery, hybrid resorbable devices can be manufactured that include relatively large amounts of a resorbable material.

In another non-limiting variation on a preferred embodiment, such as in a case of an acetabular revision surgery, the surgeon can modify a shape of the hybrid resorbable device with a suitable instrument, such as a powered burr, or other surgical instrumentation, so that the hybrid resorbable device is shaped to fit a particular application, or a patient's bone defect, with a goal that, in time, the resorbable material is replaced with the bone.

In one more non-limiting variation, a hybrid resorbable device comprises materials with different resorption rates. For example, a distal aspect of a femoral hip stem is partitioned into three different zones. The most distal zone would comprise a resorbable material with the shortest resorption time of the three zones. The next proximal zone would comprise a resorbable material that would be resorbed the next fastest, and, finally, the most proximal zone would comprise a material with a slowest rate of resorption. As the resorption takes place, the load transfer to the femur from the implant occurs more proximally, and stresses, or loads, the bone more, thereby aiding in bone growth, or remodeling. As a result, the proximal femur maintains superior bone mass.

A metal core can be utilized in certain variations of the hybrid resorbable devices, thereby providing at least two advantages to a hybrid resorbable device: first, minimization the mechanical strength that would be required from a resorbable material of the hybrid device, and, second, reduction in the amount of a mass of a resorbable materials for resorption by the body of a human or an animal.

It is to be understood that the use of the resorbable adjunct stabilization devices according to aspects and embodiments of the present invention is not limited to installation of a hip implant during revision surgery, as described above, but includes use during any joint replacement surgery. The use of the devices and methods for stabilization of prosthetic implants of bones and joints, including but not limited to hip, knee, shoulder, elbow, ankle, wrist, finger joint or toe joint, jaw, skull or spinal prostheses, further including, but not limited to, femoral head implants, femoral stem implants, acetabular cup implants, hinged knee implants, tibial, femoral, or meniscal components of knee implants, patella implants, humeral, glenoid, or ulnar components of prosthetic shoulder or elbow implants, is envisioned and falls within the scope of the present invention.

Also falling within the scope of the present invention is the use of the devices and methods according to aspects and embodiments of the present invention for treatment of various bone and joint diseases and pathological conditions, particularly in, but not limited to, disease and pathological conditions that reduce the amount and quality of bone tissue available for fitting, such as arthritis, including osteoarthritis and rheumatoid arthritis, arthropathy, avascular necrosis, cancer and metastases, tuberculosis, osteoporosis, trauma, such as fractures and dislocations, deformities, including but not limited to, birth defects and genetic anomalies, infections, such as tuberculosis, bone neoplasms, osteitis deformans, osteochondritis, osteonecrosis, bone demineralization, or any combination or variation thereof, or condition related thereto.

Use of the systems, devices, and methods for stabilization of cemented or cementless joint implants, including the joint implants stabilized by biological stabilization, is also envisioned and included within the scope of the present invention. In general, the scope of the present invention include use of the methods, devices, and systems described herein in any method of repair or attachment of bones and other tissues, such as connective tissue, including, but not limited to, bone, loose connective tissue, fibrous connective tissue, such as that found in ligaments and tendons, cartilage and adipose tissue, endothelial tissue, epithelial tissue, glandular tissue, muscle tissue, or any artificial, semi-artificial, or engineered tissue. Generally, the resorbable devices of the present invention are used wherever temporary stabilization of tissues and filling of cavities is advantageous.

Use of the systems, devices, and methods according to aspects of the present invention in conjunction with joint implant systems is not to be understood as limiting. Envisioned also are the uses of the stabilization devices described herein alone, such as, but not limited to, in for filling of cavities of bones and other tissues, and in conjunction with systems other than joint implants. Methods of manufacturing the systems and devices described herein are also envisioned.

The foregoing discloses preferred embodiments of the present invention, and numerous modifications or alterations may be made without departing from the spirit and the scope of the invention.

All documents cited herein are incorporated by reference in their entirety.

What is claimed is:

1. A system for stabilization of an implant in bone tissue of a human or an animal, comprising a prosthetic implant and a resorbable device adapted to be placed between the implant and the bone tissue, wherein the resorbable device is inserted into a cavity between the implant and the bone tissue of the human or the animal, thereby at least partially filling the cavity and reducing movements of the implant relative to the bone tissue, and wherein the resorbable device is at least partially resorbed over a predetermined period of time, thereby allowing for ingrowth of the bone tissue into the cavity.

2. The system of claim 1, wherein the resorbable device comprises a resorbable polymer.

3. The system of claim 2, wherein the resorbable polymer is poly-L-lactic acid, polyglactin acid, or a combination thereof.

4. The system of claim 2, wherein the resorbable device further comprises calcium sulphate, calcium phosphate or a combination thereof.

5. The system of claim 2, wherein the resorbable device further comprises a bioactive molecule.

6. The system of claim 5 wherein the bioactive molecule is a growth factor or antibiotic.

7. The system of claim 1, wherein the resorbable device is a spacer having a predetermined shape that at least partially fills the cavity to reduce movements of the implant relative to the bone.

8. The system of claim 1, wherein the resorbable device is inserted during a joint replacement surgery.

9. The system of claim 8, wherein the prosthetic implant is a hip implant, a knee implant, a shoulder implant, or an elbow implant, or a component thereof.

10. The system of claim 9, wherein the prosthetic implant or the component thereof is inserted into an intramedullary canal of a tubular bone, and wherein the resorbable device is inserted into the cavity between the bone and the prosthetic implant.

11. The system of claim 10, further comprising an orthopedic cable, wherein the cable is tightened around the bone, thereby tightening the bone around the resorbable device and tightening the resorbable device against the prosthetic implant or the component thereof.

12. The system of claim 10, further comprising an allograft bone, resorbable granules, or a combination thereof, wherein the allograft bone or the resorbable granules, or the combination thereof, are inserted into the cavity.

13. The system of claim 10, wherein the joint replacement surgery is revision surgery.

14. The system of claim 10, wherein the implant is a hip replacement comprising a femoral stem, wherein the femoral stem is inserted into the femoral canal, and wherein the resorbable device is inserted into the cavity between a cortex of the femur and the femoral stem of the hip implant.

15. A method for stabilization of an implant in a human or an animal, comprising: inserting the implant;
inserting a resorbable device adapted to be placed between the implant and the bone tissue, wherein the resorbable device is inserted into a cavity between the implant and the bone tissue of the human or the animal, thereby at least partially filling the cavity and reducing movements of the implant relative to the bone tissue, and wherein the resorbable device is at least partially resorbed over a predetermined period of time, thereby allowing for ingrowth of the bone tissue into the cavity.

16. The method of claim 16, wherein the resorbable device comprises a resorbable polymer.

17. The method of claim 15, wherein the resorbable device is inserted during a joint replacement surgery.

18. The method of claim 17, wherein the prosthetic implant is a hip implant, a knee implant, a shoulder implant, or an elbow implant, or a component thereof.

19. The method of claim 15, wherein the device is a spacer having a predetermined shape that at least partially fills the cavity to reduce movements of the implant relative to the bone.

20. The method of claim 18, wherein the prosthetic implant or the component thereof is inserted into an intramedullary canal of a tubular bone, and wherein the resorbable device is inserted into the cavity between the bone and the implant.

21. The method of claim 20, further comprising, after inserting the device, tightening an orthopedic cable around the bone, thereby tightening the bone around the device and tightening the device against the prosthetic implant or the component thereof.

22. The method of claim 17, wherein the joint replacement surgery is revision surgery.

23. The method of claim 20, wherein the implant is a hip replacement comprising a femoral stem, wherein the femoral stem is inserted into a femoral canal, and wherein the resorbable device is inserted into the cavity between a proximal a cortex of the femur and the femoral stem of the implant.

24. A hybrid resorbable device for stabilization of a prosthetic implant in the bone tissue of a human or an animal, comprising at least one resorbable component and at least one non-resorbable component, wherein the at least one resorbable component is at least partially inserted into the bone tissue, thereby reducing movements of the implant relative to the bone tissue, and wherein the resorbable component is at least partially resorbed over a predetermined period of time, thereby allowing for ingrowth of the bone tissue into a space from which the resorbable component has been resorbed.

25. The hybrid resorbable device of claim 24, wherein the resorbable component comprises a resorbable polymer.

26. The hybrid resorbable device of claim 25, wherein the resorbable polymer is poly-L-lactic acid, polyglactin acid, or a combination thereof.

27. The hybrid resorbable device of claim 25, wherein the resorbable device further comprises a bioactive molecule.

28. The hybrid resorbable device of claim 27, wherein the bioactive molecule is a growth factor or antibiotic.

29. The hybrid resorbable device of claim 24, wherein the hybrid resorbable device is an elongated member.

30. The hybrid resorbable device of claim 29, wherein the hybrid resorbable device is a peg, comprising a locking shoulder portion, and a peg portion, and wherein the non-resorbable component is the locking shoulder portion, and the resorbable component is the peg portion.

31. The system of claim 24, wherein the resorbable device is inserted during a joint replacement surgery.

32. The system of claim 31, wherein the implant is a hip implant, a knee implant, a shoulder implant, or an elbow implant.

33. A prosthetic implant system, comprising:
an implant; and
a hybrid resorbable device for stabilization of a prosthetic implant in the bone tissue of a human or an animal, comprising at least one resorbable component and at least one non-resorbable component, wherein the at least one resorbable component is at least partially inserted into the bone tissue, thereby reducing movements of the implant relative to the bone tissue, and wherein the resorbable component is at least partially resorbed over a predetermined period of time, thereby allowing for ingrowth of the bone tissue into a space from which the resorbable component has been resorbed.

34. The prosthetic implant system of claim 33, wherein the resorbable component comprises a resorbable polymer.

35. The prosthetic implant system of claim 34, wherein the resorbable polymer is poly-L-lactic acid, polyglactin acid, or a combination thereof.

36. The prosthetic implant system of claim 34, wherein the resorbable device further comprises a bioactive molecule.

37. The prosthetic implant system of claim 36 wherein the bioactive molecule is a growth factor or antibiotic.

38. The prosthetic implant system of claim 33, wherein the hybrid resorbable device is an elongated member.

39. The prosthetic implant system of claim 33, wherein the resorbable device is inserted during a joint replacement surgery.

40. The prosthetic implant system of claim 39, wherein the implant is a hip implant, a knee implant, a shoulder implant, or an elbow implant, or a component thereof.

41. The prosthetic implant system of claim 40, wherein the implant is an acetabular component of a hip implant comprising openings, and the hybrid resorbable device is inserted through the openings into the bone.

42. A method of installing a prosthetic implant in a body of a human or an animal, comprising:
inserting the implant into a body of the human or the animal; and
stabilizing the implant in the body of the human or the animal with a hybrid resorbable device for stabilization of the implant in a human or an animal, comprising one or more resorbable component and one or more non-resorbable component, wherein the resorbable component is at least partially inserted into a tissue of the human or the animal, thereby reducing movements of the implant relative to the tissue, and wherein the resorbable component is at least partially resorbed over a predetermined period of time, thereby allowing for ingrowth of the tissue into the cavity.

43. The method of claim 42, wherein the resorbable component comprises a resorbable polymer.

44. The method of claim 43, wherein the resorbable polymer is poly-L-lactic acid, polyglactin acid, or a combination thereof.

45. The method of claim 43, wherein the resorbable device further comprises a bioactive molecule.

46. The method of claim 46, wherein the bioactive molecule is a growth factor or antibiotic.

47. The method of claim 42, wherein the hybrid resorbable device is an elongated member.

48. The method of claim 42, wherein hybrid resorbable device of claim 29, wherein the hybrid resorbable device is a peg, comprising a locking shoulder portion, and a peg portion, and wherein the non-resorbable component is the locking shoulder portion, and the resorbable component is the peg portion.

49. The method of claim 42, wherein the resorbable device is inserted during a joint replacement surgery.

50. The method of claim 49, wherein the implant is a hip implant, a knee implant, a shoulder implant, or an elbow implant.

51. The method of claim 49, wherein the implant is an acetabular component of a hip implant, comprising openings, and the hybrid resorbable device is inserted through the openings into the bone.
